(19) United States
(12) Patent Application Publication
(10) Pub. No.: US 2018/0192916 A1
(43) Pub. Date: Jul. 12, 2018

(54) IMAGING SYSTEM FOR DIAGNOSING PATIENT CONDITION

(71) Applicant: General Electric Company, Schenectady, NY (US)

(72) Inventors: Bruno Kristiaan Bernard De Man, Clifton Park, NY (US); Prem Venugopal, Clifton Park, NY (US)

(21) Appl. No.: 15/402,386

(22) Filed: Jan. 10, 2017

(51) Int. Cl. A61B 5/107; A61B 5/103; A61B 5/021; A61B 8/06; A61B 6/00; A61B 5/055

(52) U.S. Cl. CPC A61B 5/107; A61B 5/103; A61B 5/055; A61B 8/06; A61B 6/504; A61B 5/021

(57) ABSTRACT

A system and method of characterizing a blood vessel. An imaging modality is utilized to take an image of the blood vessel that is under a first stimulated condition and an image of the blood vessel is under a second stimulated condition. The first and second images of the blood vessel are then compared to one another to determine stress and strain data of the blood vessel in order to characterize the blood vessel.

### FIELD

The subject matter described herein relates to medical imaging systems.

### BACKGROUND

Health conditions related to blood vessels typically are serious in nature. For example, an aneurysm is a localized blood-filled bulge in a blood vessel that can lead to serious health conditions, including death. In particular, brain aneurysms have a risk of bursting or rupturing, leading to hemorrhagic stroke, permanent nerve damage, or death. While aneurysms can be treated, these interventions also carry significant risk. Even small errors during surgery or procedures can similarly lead to these serious health conditions. As a result, in the case of small aneurysms, the risk from interventions is approximately the same as the rupture risk. Therefore, it is desired to better estimate the aneurysm rupture risk for this group and only intervene in patients with a high rupture risk.

Currently, rupture risk is estimated primarily based on aneurysm morphology and patient-specific factors. In particular, a patient's family history and the size of the aneurysm are the main factors in determining whether an invasive high risk procedure needs to be undertaken to save an individual's life. In the case of small aneurysms, the rupture risk and the risk of complications from an invasive procedure are approximately the same. Therefore, a need in the art exists for equipment and methods to better assess the risks associated with aneurysms and other health conditions related to individual's blood vessels.

### BRIEF DESCRIPTION

In one embodiment, a system is provided having an imaging device configured to take a first image of a blood vessel. The imaging device is also configured to take a second image of the blood vessel. One or more processors are configured to receive the first and second images and compare the first and second images to determine a characteristic of the blood vessel based on the first and second image.

In one embodiment, a method of characterizing a blood vessel is provided. In this method the blood vessel is stimulated. An image of the blood vessel is taken with imaging modality when the blood vessel is stimulated and is also taken when the blood vessel is not stimulated. The image taken of the blood vessel when stimulated is then compared to the image taken of the blood vessel when not stimulated to characterize the blood vessel.

In another embodiment, a method is provided for characterizing a blood vessel. In this method the blood vessel is stimulated. An image is taken of an aneurysm of the blood vessel during a first condition after the blood vessel is stimulated and an image is similarly taken of the aneurysm of the blood vessel during a second condition after the blood vessel is stimulated. The image of the aneurysm of the blood vessel taken during the first condition is compared to the image of the aneurysm of the blood vessel taken during the second condition to determine the likelihood the aneurysm will rupture.

### BRIEF DESCRIPTION OF THE DRAWINGS

FIG. 1 is a schematic diagram of a system for determining a characteristic of a blood vessel;

FIG. 2 illustrates a sample image generated by the system for determining a characteristic of a blood vessel;

FIG. 3 illustrates a sample image generated by the system for determining a characteristic of a blood vessel;

FIG. 4 illustrates a flow chart of a method of characterizing a blood vessel;

FIG. 5 illustrates a cross-sectional image of a vessel that is not stimulated and a cross-sectional image of the vessel in the same location, but with the vessel stimulated;

FIG. 6 illustrates another example of a cross-sectional image of a vessel that is not stimulated and a cross-sectional image of the vessel in the same location, but with the vessel stimulated;

FIG. 7 illustrates an example of a cross-sectional image of a lesion that is not stimulated and a cross-sectional image of the lesion in the same location, but with the lesion stimulated; and

FIG. 8 illustrates a cross-sectional image of a lesion that is not stimulated and a cross-sectional image of the lesion in the same location, but with the lesion stimulated.

### DETAILED DESCRIPTION

Blood vessel conditions such as aneurysms can be treated by endovascular or surgical means. However, the morbidity and mortality rate of these procedures is comparable to the rupture risk itself, especially for small aneurysms. Thus, often the best treatment for an aneurysm can be no treatment at all. The system and method disclosed more accurately predicts characteristics of blood vessels, including aneurysm rupture risk. Therefore, in the case of such an aneurysm, only those patients with a high rupture risk are considered for intervention. Thus, greater preventive care is provided to the patient.

A significant amount of research has focused on analyzing the fluid dynamic properties of the blood flow inside aneurysm with the ultimate aim of including it as part of the rupture risk. These studies have found correlation between growth/rupture and aspects such as unstable jets, low wall shear stress, or resonance phenomena. Histochemical studies have also been conducted, relating structural features to aneurysm rupture risk.

By utilizing the system and method provided, aneurysm rupture risk is assessed based on vaso-dilation/constriction. Imaging modality such as a computed tomography (CT) device, an x-ray device, a magnetic resonance imaging (MRI) device, an ultrasound device, or the like, can utilized to image a stimulated blood vessel. These imaging modalities are used to non-invasively visualize internal anatomical structures or functional properties of the human body. Radiography and computed tomography use x-rays to image various tissues based on their amount of x-ray attenuation or the energy-dependence thereof. MRI applies a strong external magnetic field and images how different tissues respond to this external field. Ultrasonic imaging sends out acoustic waves to image the ultrasonic reflection properties of different tissues and organ boundaries.

Using such an imaging modality, the blood vessel is stimulated by one or more methods, such as by providing a patient with one or more of a vaso-dilating agent or a vaso-constricting agent. By imaging the aneurysm at two or more phases of dilation/constriction, an estimate of the local compliance can be made. For instance, if the aneurysm wall is devoid of smooth muscle cells in a certain location, it is more likely to change shape or volume due to vaso-dilation or vaso-constriction.

The images can be compared to one another either manually by a radiologist that looks at the same anatomy and evaluates the impact of the vaso-dilation or vaso-constriction stimulation on the aneurysm, or by using one or more processors that utilize a computer algorithm. In particular, the one or more processors determine resulting strain caused by the stimulation and combined with other parameters or characteristics of the aneurysm and patient computes a risk of rupture of the aneurysm. This is provided either as a percentage or a score that indicates rupture risk for the patient.

In general, the computer algorithm can perform the steps of (1) registering the image of the vessel/aneurysm/lesion before stimulation and the image of the vessel/aneurysm/lesion after stimulation, (2) estimating the local displacement vectors of specific portions of the vessel/aneurysm/lesion and (3) estimating the local strain and elasticity of specific portions of the vessel/aneurysm/lesion.

The calculations that are determined by the one or more processors with the algorithm can include fluid dynamic calculations using multi-phase images. The fluid dynamic model parameters can be tuned to match the images. In addition, the fluid dynamic calculations can yield additional information for diagnosing rupture risk including aneurysm wall stress, which can be combined with other parameters and information extracted as a result of the images.

In addition to the aneurysm, the method and system can image a parent vessel without the administration of a stimulus such as a vasodilator/vasoconstrictor. The parent vessel strain rate is determined and is used as a parameter or characteristic to normalize the strain rate obtained for the aneurysm. The normalized strain rate can then be used to predict rupture risk. Optionally, distensibility/compliance of the aneurysm wall as well as the parent vessel based on the measurements can be utilized, where:

Distensibility is defined as:

$$D = \frac{1}{A} \frac{dA}{dP}$$

And compliance is defined as:

$$C = \frac{dA}{dP}$$

where A represents the cross-sectional area of an imaged vessel and P represents the pressure of fluid within the vessel.

Unlike a healthy vessel, where cross-sectional area is typically defined on a plane normal to centerline, numerous definitions are possible for a saccular aneurysm. The change in cross-sectional area, either due to vasodilator/vasoconstrictor introduction or due to change in blood pressure, can be determined from the images. The changes in blood pressure can be determined non-invasively based on cuff measurements or it could be combined with a fluid dynamics model to predict blood pressure changes at the aneurysm site. The distensibility and compliance estimates can also be normalized based on the same quantities computed for the parent vessel.

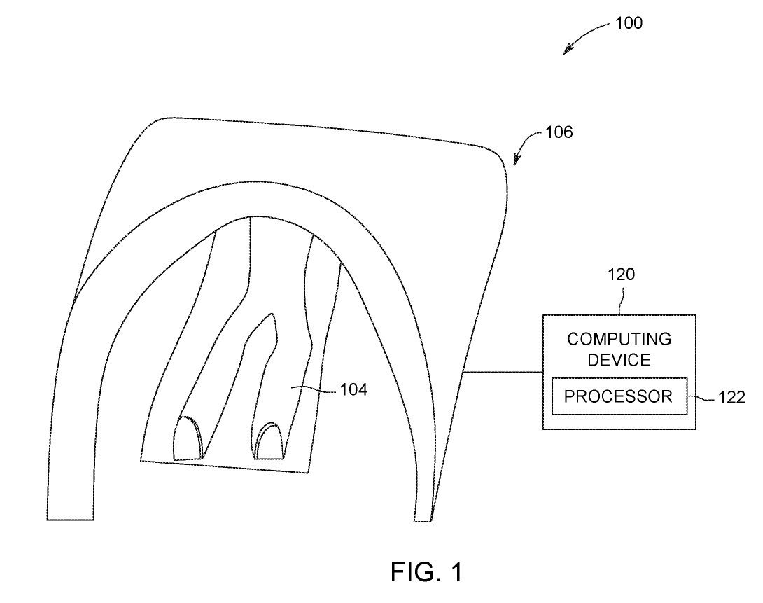

FIG. 1 shows a schematic diagram showing features of an imaging system 100 for characterizing a blood vessel 102 of a patient 104. The system 100 includes an imaging device 106 that takes real-time images 107 of the blood vessel 102. The imaging device 106 can be any type, including but not limited to CT, X-ray, MRI, Ultrasound and the like. The imaging device 106 takes multiple images 107 of the blood vessel 102 for analysis. In one embodiment, the images 107 are cross-sectional images of the blood vessel 102.

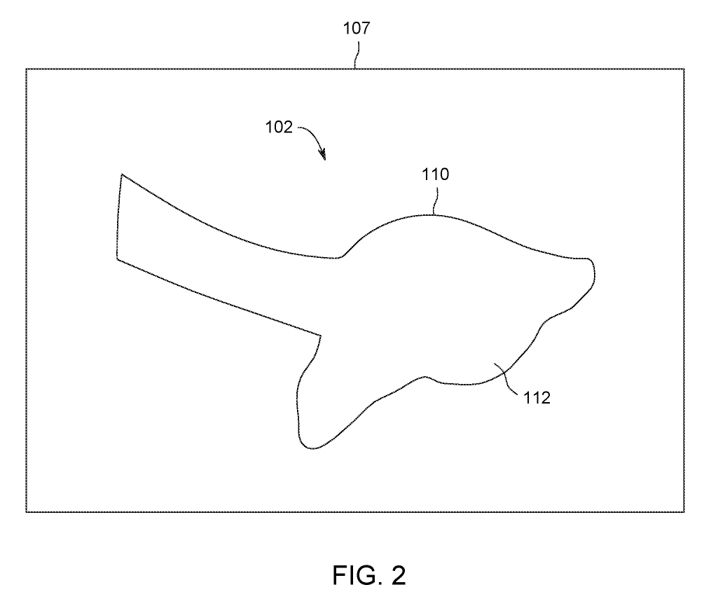

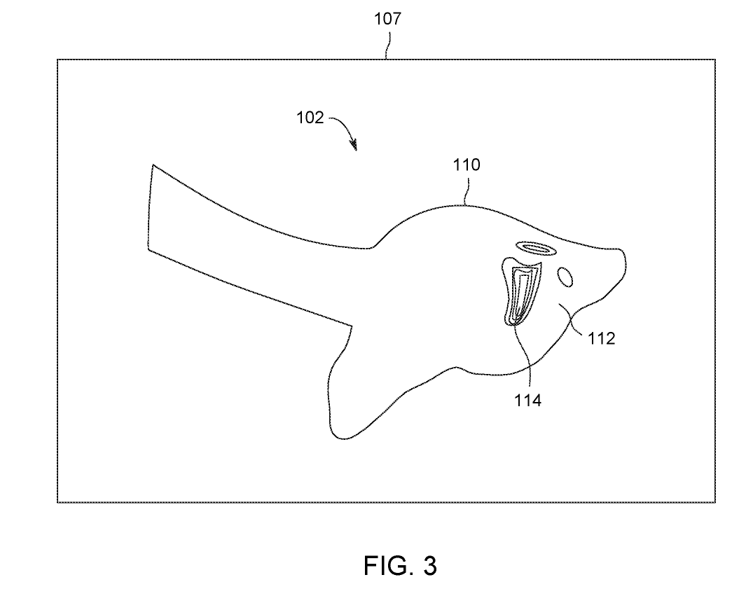

FIGS. 2 and 3 show example images 107 of a blood vessel 102 that contains an aneurysm 110 of the patient 104 and the amount of strain on the wall 112 of the blood vessel 102 including the aneurysm 110. In FIG. 2, the blood vessel is shown in a first condition, which in this figure is a normal or steady state in which a stimulus is not presented. FIG. 3 shows the same blood vessel and aneurysm taken at a different point of time or during a second condition when the blood vessel 102 is stimulated and an increased strain area 114 is provided. While FIG. 2 is shown first and described as a first condition and FIG. 3 shown second and is described as a second condition, the order in which images 107 are taken does not vary the comparison methodology described herein. Similarly, while FIG. 2 shows an image 107 that presents a blood vessel 108 and aneurysm 110 that is in an unstimulated, steady state, the first condition of the blood vessel 108 and aneurysm 110 can also present an image 107 of a stimulated blood vessel 108 and aneurysm 110 that either has enhanced or reduced stimulation as compared to the image 107 of the second condition. In particular, for the comparison methodology, the blood vessel 108 and aneurysm 110 need only be under differing levels of displacement from the first condition to the second condition so that differing strain imagery and data is obtained from the imaging device 106 for comparison and analysis.

The blood vessel, and consequently the aneurysm, can be stimulated in any manner, including through use of medication or agent that is ingested or administered to the patient. This includes, but is not limited to a vaso-dilation agent administered to temporarily dilate or relax the blood vessel or a vaso-constriction agent administered to temporarily constrict or tighten the blood vessel. Alternatively, other methods are utilized to stimulate the blood vessel and aneurysm and include, but are not limited to, having the patient exercise, having the patient use compression clothing, movement of a body part of a patient, or the like.

By imaging and comparing the aneurysm in two or more different phases of stimulation, the structural integrity of the aneurysm can be assessed. In particular, the images and stress and strain data derived therefrom are used to determine if the walls of an aneurysm are devoid or lacking of smooth muscle cells in any of the regions of the aneurysm. The lack of smooth muscle cell indicates a higher likelihood of rupture. Because vasodilators/vasoconstrictors/stimuli work by relaxing/constricting smooth muscle cells, the response of the aneurysm wall to such agents correlates to the presence or absence of smooth muscle cells and therefore the rupture risk of the aneurysm is obtained.

A remote computing device 120 optionally is presented as part of the system. The computing device 120 includes one or more processors 122 that are able to either communicate with the imagining device 106 or receive inputs related to the images 107 taken by the imaging device. The one or more processors 122 can have an algorithm therein such as a deep learning algorithm to compare the images and/or utilize historical data, blood flow data including the size and position of an aneurysm in a blood vessel, and the like, including but not limited to additional images of the blood vessel of the patient, parent blood vessels, or of blood vessels of other patients. From the imagery and other data received the one or more processors 122 determine the likelihood of a medical or health condition of the blood vessel. This includes the analysis of stress, strain data, size data, and position data in the blood vessel of an aneurysm to identify smooth cell deficiencies in the walls of the aneurysm and to form a fluid dynamic model of the aneurysm utilizing an algorithm.

The images of the aneurysm and blood vessel acquired under the stimulated or unstimulated condition could also be used to generate a model of blood flow within the aneurysm. By modeling the fluid dynamic properties of the blood flow inside an aneurysm the model can identify aspects such as unstable jets, low wall shear stress, or resonance phenomena that correlate and are related to a high probability of a rupture of the aneurysm. The additional information from the fluid dynamic calculations are combined with aneurysm wall strain/stress by the one or more controllers to determine rupture probability of the aneurysm. This also includes providing a present time rupture percentage or a score indicating the risk level, including ranges such as moderate, severe and critical.

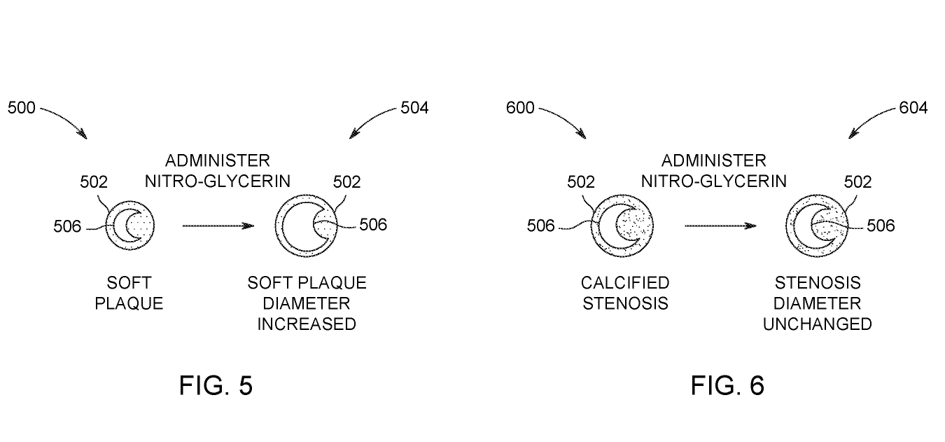

FIGS. 5 through 8 illustrate examples of comparing images of a vessel to diagnose or monitor a condition of a vessel. FIG. 5 illustrates a cross-sectional image 500 of a vessel 502 that is not stimulated and a cross-sectional image 504 of the vessel 502 in the same location, but with the vessel stimulated (such as by providing nitro-glycerine to the patient). The image 500 may be referred to as a non-stimulated image while the image 504 can be referred to as a stimulated image. One or more processors 122 of the remote computing device 120 may receive these images 500, 504, and determine and compare characteristics of the images 500, 504 to diagnose or monitor one or more conditions or states of the vessel 502. For example, the processors 122 may measure diameters of the vessel 502 in the images 500, 504 and/or sizes (e.g., areas) of an object 506 in the vessel 502 in the images 500, 504. The processors 122 can compare the diameters and the sizes of the object 506 to determine that both the diameter of the vessel 502 and the size of the object 506 in the vessel 502 increased following stimulation of the vessel 502. The processors 122 can then determine (based on a memory structure, such as a lookup table or database) that the increase in diameter and/or the increase in size of the object 506 indicate that the object 506 is soft or vulnerable plaque.

FIG. 6 illustrates a cross-sectional image 600 of a vessel 502 that is not stimulated and a cross-sectional image 604 of the vessel 502 in the same location, but with the vessel stimulated (such as by providing nitro-glycerine to the patient). The image 600 may be referred to as a non-stimulated image while the image 602 can be referred to as a stimulated image. One or more processors 122 of the remote computing device 120 may receive these images 600, 604, and determine and compare characteristics of the images 600, 604 to diagnose or monitor one or more conditions or states of the vessel 502. For example, the processors 122 may measure diameters of the vessel 502 in the images 600, 604 and/or sizes (e.g., areas) of the object 504 in the vessel 502 in the images 600, 604. The processors 122 can compare the diameters and the sizes of the object 504 to determine that the diameter of the vessel 502 and the size of the object 504 in the vessel 502 did not change following stimulation of the vessel 502. The processors 122 can then determine (based on a memory structure, such as a lookup table or database) that the lack of change in diameter and/or in the size of the object 504 indicates that the vessel 502 is diseased or has inflammation or has calcified stenosis.

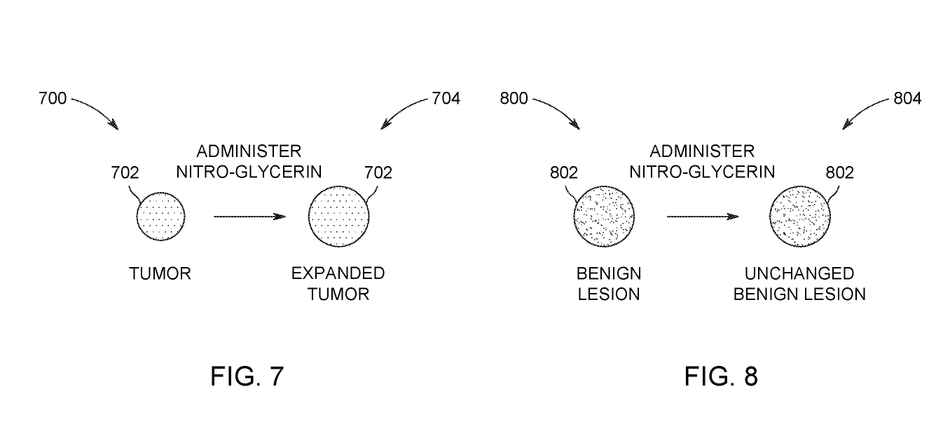

The processors 122 may examine images of one or more lesions in a patient in order to determine the size of the lesion, the number of blood vessels in the lesion, the size of the blood vessels, and/or the flexibility of the vessels and the surrounding tissues. These characteristics may be used to diagnose and/or monitor tumors in the patient. FIG. 7 illustrates a cross-sectional image 700 of a lesion 702 that is not stimulated and a cross-sectional image 704 of the lesion 702 in the same location, but with the lesion stimulated (such as by providing nitro-glycerine to the patient). The image 700 may be referred to as a non-stimulated image while the image 702 can be referred to as a stimulated image.

The one or more processors 122 of the remote computing device 120 may receive these images 700, 704, and determine and compare characteristics of the images 700, 704 to diagnose or monitor one or more conditions or states of the patient. For example, the processors 122 may measure diameters of the lesion 702 in the images 700, 704. The processors 122 can compare the diameters to determine that the diameter of the lesion 702 has increased from stimulation of the lesion 702. The processors 122 can then determine (based on a memory structure, such as a lookup table or database) that this increase in diameter indicates that the lesion 702 has a certain level of vascularity and hemodynamic properties and is malignant.

FIG. 8 illustrates a cross-sectional image 800 of a lesion 802 that is not stimulated and a cross-sectional image 804 of the lesion 802 in the same location, but with the lesion stimulated (such as by providing nitroglycerine to the patient). The image 800 may be referred to as a non-stimulated image while the image 802 can be referred to as a stimulated image.

The one or more processors 122 of the remote computing device 120 may receive these images 800, 804, and determine and compare characteristics of the images 800, 804 to diagnose or monitor one or more conditions or states of the patient. For example, the processors 122 may measure diameters of the lesion 802 in the images 800, 804. The processors 122 can compare the diameters to determine that the diameter of the lesion 802 has not changed or has not increased from stimulation of the lesion 802. The processors 122 can then determine (based on a memory structure, such as a lookup table or database) that this lack of an increase in diameter indicates that the lesion 802 has a certain level of vascularity and hemodynamic properties and is benign.

As another example, the processors 122 may examine changes in perfusion behavior of a vessel in one or more non-stimulated images and one or more stimulated images to assist in the diagnosis or monitoring in changes of a brain or other organ of the patient.

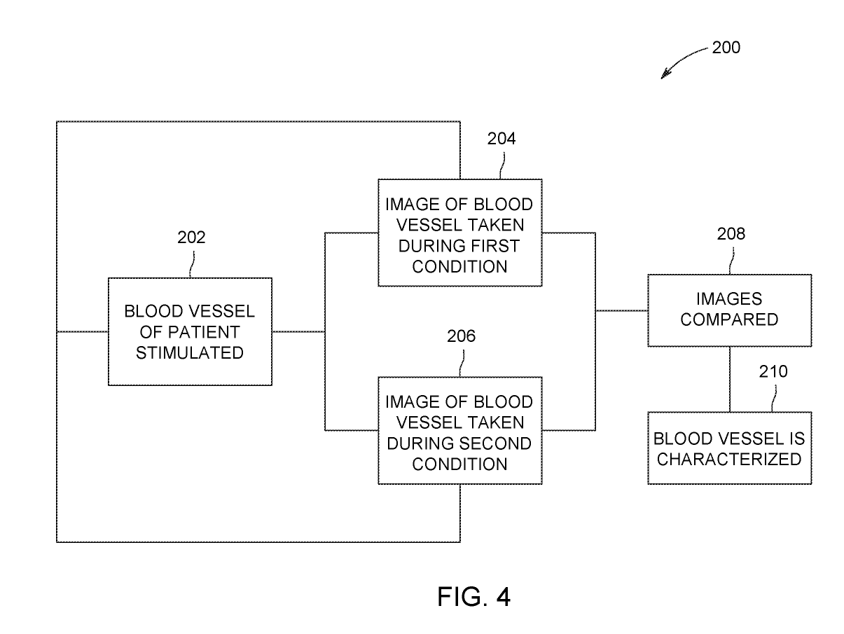

FIG. 4 shows a flowchart of a method 200 for characterizing a blood vessel. The method 200 can represent operations performed by the processors 122 described above, such as operations performed by the processors 122 operating based on instructions included or provided by a software application. Optionally, the method 200 can represent an algorithm that is used to create such a software application. At 202, a blood vessel of a patient is stimulated. This stimulation can involve providing the patient with one or more medications, such as nitro-glycerin. At 204, an image of the stimulated blood vessel is taken during a first condition. At 206, an image of the blood vessel is taken during a second condition. This second condition may be non-stimulation of the blood vessel. In one embodiment, the non-stimulated image of the vessel is obtained prior to the stimulated image. Alternatively, the stimulated image is obtained before the non-stimulated image (e.g., after stimulation of the vessel ceases). At 208, the image of the stimulated blood vessel taken during the first condition is compared to the image of the stimulated blood vessel taken during the second condition. The processors 122 can determine characteristics of the vessel and/or objects in the vessel from these images. At 210, a determination is made based on the comparison of the images characterizing the blood vessel. For example, the processors 122 can compare the characteristics of the vessel and/or objects in the vessel to diagnose the presence of vulnerable plaque, stenosis, tumors, or benign lesions.

In one or more embodiments of the inventive subject matter described herein, a state of a blood vessel is transformed from a non-stimulated state to a stimulated state, with images acquired of the vessel in each state. The images of the same vessel in the different states are compared (or at least characteristics of the vessel in the different states are determined and compared) in order to identify or diagnose a condition of the patient. One or more responsive actions may be implemented based on this diagnosis, such as administering a medical treatment or medication based on the diagnosis.

While described in the context of characterizing a blood vessel for risk associated with aneurysm rupture, the imaging and comparison methodology can be utilized in other embodiments to similarly characterize health related conditions within a blood vessel. In one example, the methodology is utilized in diagnosing stenosis and vulnerable plaque. If a vaso-dilation agent such as nitro-glycerine is administered as a stimulus to a blood vessel with stenosis and the stenosis diameter has little to no change, the blood vessel can be characterized as having a high probability of disease or inflammation or calcification. In another example, the methodology is utilized in tumor diagnosis or therapy. In particular, by monitoring a number of blood vessels, their size, and the flexibility of the vessels and the surrounding tissues tumor identification and characterization is provided. Similarly, changes in perfusion behavior after administration of vessel-dilators/constrictors can be an additional indicator for brain imaging that can be detected and characterized using this methodology.

As an example of the method and system, a patient who has been diagnosed as having a brain aneurysm has an initial CT scan without any stimulus provided. The CT scan provides an image of the aneurysm during a normal functioning of the blood vessel. The patient is then administered a vaso-dilation agent to relax the blood vessel. A second CT scan is provided and an image of the aneurysm is taken while the blood vessel is dilated. The physician compares the initial steady state image to the image taken while the blood vessel is dilated and recognizes that the walls of the aneurysm do not appear to be under a great amount of strain and the aneurysm is characterized as not having a high risk of rupture. Then, based on the size, shape and lack of indicated strain the physician decides at the time surgery is not required.

In yet another example, a patient who has been diagnosed as having a heart aneurysm is provided a vaso-constriction agent prior to having an MRI examination preformed. The MRI produces an image of the aneurysm while the blood vessel is stimulated, in this case restricted. After a period of time, the MRI takes a second image of the aneurysm after the effects of the vaso-constriction agent is reduced. The images are communicated to one or more processors of a computing device that compares the images using an algorithm to provide a fluid dynamic model of the images. In addition, blood flow data is taken and inputted into the one or more processors. Based on the images, historical data related to the patient's age, an increase in diameter of the aneurysm, the blood flow data and consequential model, the one or more processors characterize the aneurysm as having a high risk of rupturing and the patient undergoes evasive surgery on the aneurysm.

In another example, a patient has an ultrasound performed to address chest pains and sluggishness. An initial ultrasound image is formed of the blood vessels around the heart. The patient then exercises for 30 minutes under the observation of medical staff. A second ultrasound is then administered and a second image is taken. The images are inputted into a computing device that has one or more processors that compare the images that indicate increased strain in one of the blood vessels as a result of increase blood pressure and the one or more processors characterize the blood vessel as having a higher than normal probability of stenosis.

In yet another example, a patient who has been diagnosed as having a brain aneurysm is provided a vaso-constriction agent prior to having a CT scan examination preformed. The CT scan produces an image of a region of interest (ROI) of the aneurysm while the blood vessel is stimulated, in this case restricted. The patient is then administered a vaso-dilation agent after the initial CT scan, but before undergoing a second CT scan. The patient then undergoes a second CT scan of the ROI of the aneurysm to take a second image of the ROI of the aneurysm, this time with the aneurysm dilated. A physician then compares the images and recognizes multiple areas in the ROI show significant strain. The physician then inputs the images into a computing device that has one or more processors that also compare the images and characterize the aneurysm as having a high probability of rupture. As a result, surgery is scheduled to address the aneurysm.

In one embodiment, a method of characterizing a blood vessel is provided. Steps include, stimulating the blood vessel, taking an image of the blood vessel when stimulated with an imaging modality and taking an image of the blood vessel when not stimulated with the imaging modality. The image taken of the blood vessel when stimulated is then compared to the image taken of the blood vessel when not stimulated to characterize the blood vessel. In one embodiment, the step of stimulating the blood vessel comprises administering an agent that dilates the blood vessel. In another embodiment, the agent is a vaso-dilator.

In one embodiment, the step of stimulating the blood vessel comprises administering an agent that constricts the blood vessel. In another embodiment, the agent is a vaso-constrictor. In another embodiment, the step of stimulating the blood vessel comprises one of exercising, using compression clothing, intaking of a medication, or movement of a body part of a patient.

In one embodiment, the imaging modality is one of a CT scan, MRI or ultrasound. In another embodiment, the image taken of the blood vessel when stimulated is taken before the image taken of the blood vessel when not stimulated. In yet another embodiment, the image taken of a lesion when not stimulated is taken before the image of the lesion when stimulated.

In one embodiment, the blood vessel has an aneurysm. In another embodiment, an additional step of predicting an increase in the likelihood the aneurysm will rupture compared to the likelihood of rupture without the method is provided. In yet another embodiment, one or more processors are configured to use a deep learning algorithm to compare the image of the blood vessel when stimulated to the image of the blood vessel when not stimulated and predict the increase in the likelihood the aneurysm will rupture compared to the likelihood of rupture without the method.

In one embodiment, the characteristics of the blood vessel comprise a geometrical configuration of the blood vessel. In another embodiment, the condition of the patient is determined by registering the non-stimulated image and the stimulated image, estimating local displacement vectors of portions of the blood vessel, and estimating local strain and elasticity of the portions of the blood vessel.

In one embodiment, a method of characterizing a blood vessel is provided. Steps include stimulating the blood vessel, taking an image with an imaging modality of an aneurysm of the blood vessel during a first condition after the blood vessel is stimulated, and taking an image with the imaging modality of the aneurysm of the blood vessel during a second condition after the blood vessel is stimulated. The image of the aneurysm of the blood vessel taken during the first condition is then compared to the image of the aneurysm of the blood vessel taken during the second condition to determine the likelihood the aneurysm will rupture.

In one embodiment, the likelihood the aneurysm will rupture is determined based on the diameter of the aneurysm. In another embodiment, the likelihood the aneurysm will rupture is determined based on an amount of strain detected on a portion of the aneurysm. In yet another embodiment, there is a reduction in the effect of stimulating the blood vessel from the first condition to the second condition.

In one embodiment, a system is provided. The system has an imaging device configured to take a first image of a blood vessel. The imaging device is configured to take a second image of the blood vessel. One or more processors are configured to receive the first and second images. The one or more processors also are configured to compare the first and second images and determine a characteristic of the blood vessel based on the first and second image.

In one embodiment, the one or more processors are also configured to determine the characteristic of the blood vessel based on historical data related to the blood vessel. In another embodiment, the one or more processors use an algorithm to form a fluid dynamic model to determine the characteristic of the blood vessel. In yet another embodiment, the imagine device is at least one of CT scan, MRI or ultrasound.

As used herein, an element or step recited in the singular and proceeded with the word “a” or “an” should be understood as not excluding plural of said elements or steps, unless such exclusion is explicitly stated. Furthermore, references to “one embodiment” of the presently described subject matter are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the subject matter set forth herein without departing from its scope. While the dimensions and types of materials described herein are intended to define the parameters of the disclosed subject matter, they are by no means limiting and are exemplary embodiments. Many other embodiments will be apparent to those of skill in the art upon reviewing the above description. The scope of the subject matter described herein should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the subject matter set forth herein, including the best mode, and also to enable a person of ordinary skill in the art to practice the embodiments of disclosed subject matter, including making and using the devices or systems and performing the methods. The patentable scope of the subject matter described herein is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A system comprising:
an imaging device configured to generate a non-stimulated image of a blood vessel in a patient; and
one or more processors configured to determine one or more characteristics of the blood vessel from the non-stimulated image,
wherein, responsive to a state of the blood vessel transforming from a non-stimulated state to a stimulated state, the imaging device is configured to generate a stimulated image of the blood vessel and the one or more processors are configured to determine one or more characteristics of the blood vessel from the stimulated image, the one or more processors also configured to determine a condition of the patient based on the one or more characteristics of the blood vessel determined from the non-stimulated image and the one or more characteristics of the blood vessel determined from the stimulated image.

2. The system of claim 1, wherein the state of the blood vessel is transformed by administering an agent that dilates or constricts the blood vessel of the patient

3. The system of claim 1, wherein the imaging device includes one or more of a computed tomography (CT) imaging device, a magnetic resonance imaging (MRI) device, an x-ray imaging device, or an ultrasound imaging device.

4. The system of claim 1, wherein the one or more processors are configured to determine the condition of the patient by identifying an aneurysm in the blood vessel based on a size of the blood vessel increasing in the stimulated state from the non-stimulated state.

5. The system of claim 1, wherein the one or more processors are configured to determine the condition of the patient by identifying at least one of presence of disease, presence of inflammation, or presence of plaque in the blood vessel responsive to both a size of the blood vessel and a size of an imaged object in the blood vessel increasing from the non-stimulated state to the stimulated state.

6. The system of claim 1, wherein the one or more processors are configured to determine the condition of the patient by identifying at least one of presence of disease, presence of inflammation, or presence of plaque in the blood vessel responsive to a size of the blood vessel and a size of an imaged object in the blood vessel failing to increase from the non-stimulated state to the stimulated state.

7. The system of claim 1, wherein the one or more processors are configured to determine the condition of the patient by identifying a tumor in the patient responsive to a size of a lesion increasing from the non-stimulated state to the stimulated state.

8. The system of claim 1, wherein the one or more processors are configured to determine the condition of the patient by identifying a benign lesion in the patient responsive to a size of a lesion failing to increase from the non-stimulated state to the stimulated state.

9. The system of claim 1, wherein the characteristics of the blood vessel comprise a geometrical configuration of the blood vessel.

10. The system of claim 1, wherein the condition of the patient is determined by registering the non-stimulated image and the stimulated image, estimating local displacement vectors of portions of the blood vessel, and estimating local strain and elasticity of the portions of the blood vessel.

11. A method comprising:
obtaining a non-stimulated image of a blood vessel in a patient;
determining one or more characteristics of the blood vessel from the non-stimulated image;
transforming a state of the blood vessel from a non-stimulated state to a stimulated state;
obtaining a stimulated image of the blood vessel;
determining one or more characteristics of the blood vessel from the stimulated image; and
determining a condition of the patient based on the one or more characteristics of the blood vessel determined from the non-stimulated image and the one or more characteristics of the blood vessel determined from the stimulated image.

12. The method of claim 11, wherein transforming the state of the blood vessel includes administering an agent that dilates the blood vessel to the patient.

13. The method of claim 12, wherein the agent is a vaso-dilator.

14. The method of claim 11, wherein transforming the state of the blood vessel includes administering an agent that constricts the blood vessel.

15. The method of claim 14, wherein the agent is a vaso-constrictor.

16. The method of claim 11, wherein transforming the state of the blood vessel includes one or more of exercising, using compression clothing, consuming a medication, or moving of a body part of the patient.

17. The method of claim 11, wherein the non-stimulated image and the stimulated image of the blood vessel are one or more of CT images, MRI images, x-ray images, or ultrasound images.

18. The method of claim 11, wherein determining the condition of the patient includes identifying an aneurysm in the blood vessel responsive to a size of the blood vessel increasing in the stimulated state from the non-stimulated state.

19. The method of claim 11, wherein determining the condition of the patient includes identifying vulnerable plaque in the blood vessel responsive to both a size of the blood vessel and a size of an imaged object in the blood vessel increasing from the non-stimulated state to the stimulated state.

20. A system comprising:
an imaging device configured to generate a non-stimulated image of a blood vessel in a patient; and
one or more processors configured to determine one or more characteristics of the blood vessel from the non-stimulated image,
wherein, responsive to a state of the blood vessel transforming from a non-stimulated state to a stimulated state, the imaging device is configured to generate a stimulated image of the blood vessel and the one or more processors are configured to determine one or more characteristics of the blood vessel from the stimulated image, the one or more processors also configured to determine one or more of:
a presence of an aneurysm in the blood vessel responsive to a size of the blood vessel increasing in the stimulated state from the non-stimulated state,
a presence of vulnerable plaque in the blood vessel responsive to both a size of the blood vessel and a size of an imaged object in the blood vessel increasing from the non-stimulated state to the stimulated state,
a presence of calcified stenosis in the blood vessel responsive to a size of the blood vessel and a size of an imaged object in the blood vessel failing to increase from the non-stimulated state to the stimulated state,
a presence of a tumor in the patient responsive to a size of the blood vessel increasing from the non-stimulated state to the stimulated state, or
a presence of a benign lesion in the patient responsive to a size of the blood vessel failing to increase from the non-stimulated state to the stimulated state.

21. The system of claim 20, wherein the state of the blood vessel is transformed by administering an agent that dilates or constricts the blood vessel of the patient.

22. The system of claim 20, wherein the imaging device includes one or more of a computed tomography (CT) imaging device, a magnetic resonance imaging (MRI) device, an x-ray imaging device, or an ultrasound imaging device.
